(19) United States
(12) Patent Application Publication
(10) Pub. No.: US 2022/0225908 A1
(43) Pub. Date: Jul. 21, 2022

(54) METHODS AND SYSTEMS FOR USING SURROGATE MARKERS TO IMPROVE NUTRITION, FITNESS, AND PERFORMANCE

(71) Applicant: NUEON INC., Menlo Park, CA (US)

(72) Inventors: Robert G. MESSERSCHMIDT, Menlo Park, CA (US); Hershel MACAULAY, Palo Alto, CA (US); Dara ROUHOLIMAN, San Francisco, CA (US)

(73) Assignee: NUEON INC., Menlo Park, CA (US)

(21) Appl. No.: 17/581,595

(22) Filed: Jan. 21, 2022

Related U.S. Application Data
(60) Provisional application No. 63/199,739, filed on Jan. 21, 2021.

(51) Int. Cl. A61B 5/145; A61B 5/1455; A61B 5/00

(52) U.S. Cl. CPC A61B 5/14546; A61B 5/6826; A61B 5/746; A61B 5/1455

(57) ABSTRACT

Systems, apparatuses and methods for the non-invasive measurement of a surrogate for a biomarker in a person's blood. In the example where the surrogate is a level of fat in the blood of the person and the biomarker is the level of triglycerides in the person's blood, the correlation between measurements of the surrogate and the level of triglycerides may be used to generate a recommendation to the person regarding a change in their intake of food, their exercise regimen, or another aspect of their lifestyle.

### CROSS REFERENCE

The present application claims priority to U.S. 63/199,739, filed Jan. 21, 2021, the entire disclosure of which is incorporated herein by reference.

### BACKGROUND

Conventional approaches to the delivery of health care and the treatment of disease are less than ideal in at least some respects. Over three trillion dollars is spent annually on health care in the United States, and much of this money, as well as the time and expertise of medical professionals and the supporting medicines, equipment and facilities, is directed towards reacting to symptoms and medical problems caused by preventable diseases. It would be beneficial to have improved ways of measuring and analyzing patient characteristics that would allow preventive measures to be taken, such as modifications in diet and lifestyle. Another problem with conventional approaches is that the paradigm for providing health care is based substantially around averages, yet most people are not average in at least some respects that may impact the efficacy of conventional health care methods. Although efforts have been made to personalize healthcare, the effectiveness of personalized care can be limited to the accuracy and frequency of data available for a given subject. Also, many people wish to improve their energy level, athletic performance, or appearance, and such people could benefit from improved information about their wellness and physical conditioning, even though such people may not need medical care or be at risk of disease.

Work in relation to the present disclosure suggests that prior approaches to health and wellness may have placed more reliance on biomarkers than would be ideal. For example, biomarkers may be more difficult to detect than would be ideal in at least some instances. Also, using biomarker data to generate recommendations or suggest medical treatments may result in increased regulatory compliance requirements that are not needed for effective health and wellness applications, where such applications may be associated with the decreased availability of biomarker data in at least some instances.

Work in relation to the present disclosure also suggests that it would be helpful to enable the non-invasive measurement of blood characteristics to reduce people's resistance to blood draws and similar laboratory tests. Further, work in relation to the present disclosure suggests that measurements of surrogates for biomarkers may be used to evaluate a person's health and if desired, generate recommendations to a person to make a change in their nutrition, exercise regimen, or lifestyle in order to improve their health.

In light of the above, there is a need for improved systems, devices and methods for non-invasive measurements of a person's blood that can be used to provide meaningful information regarding the health and wellness of the subject.

### SUMMARY

The presently disclosed systems, methods and apparatus enable non-invasive measurements of one or more characteristics of a person's blood. In some embodiments, the measurements are made using a transdermal near infrared sensor and associated spectroscopy components that operate to illuminate a person's skin and internal blood vessels and measure a change in the level of fat in the person's blood over a time interval. In some embodiments, a difference in the level of fat in a person's blood over a time interval is associated with a change in the level of triglycerides in the person's blood over the time interval.

In some embodiments, the near IR spectroscope and data processing methods may be used to determine a level of a surrogate for a biomarker in a person's blood. In embodiments where the surrogate is a level of fat in the blood of the person and the biomarker is the level of triglycerides in the person's blood, the correlation between measurements of the surrogate and the level of triglycerides may be used to generate a recommendation to the person regarding a change in their intake of food, their exercise regimen, or another aspect of their lifestyle.

In some embodiments, measurements of the level of fat in a person's blood at two different times may be used to determine how that level changes during a time interval. The change in the level of fat in a person's blood may be used as a surrogate for the change in the triglyceride level during that interval. The change in the triglyceride level may be used to suggest an experiment to the person to control the variation in their triglyceride level by changing their diet or another aspect of their lifestyle.

In some embodiments, the system may suggest that the person make a change to their diet, exercise regimen, or other aspect of their lifestyle and then perform blood measurements at specific times to determine the impact of that change on their triglyceride level. This can be useful information, as higher triglyceride levels and/or spikes in triglyceride levels have been found to be associated with heart disease and other ailments.

Embodiments of the disclosure provide a simple, non-invasive way for a person to monitor aspects of their health in the comfort of their home. This can enable them to make positive changes to their diet or other aspect of their lifestyle and improve their overall health. Another benefit of the system and methods described is that it can reduce the use of medical professionals and equipment for the treatment of preventable diseases, and reserve those for other important uses and patients.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

Better understanding of the features, advantages and principles of the present disclosure will be obtained by reference to the following detailed description that sets forth illustrative embodiments, and the accompanying drawings of which:

FIG. 1A is a diagram illustrating a persons' finger placed on a non-invasive apparatus comprising a source of illumination, a sensor and associated components of a near IR spectroscope for use in acquiring and processing measurements of a person's blood, in accordance with some embodiments;

FIG. 1B is a diagram illustrating a persons' finger placed on a window of a non-invasive apparatus comprising a source of illumination, a sensor, and associated components of a near IR spectroscope for use in acquiring and processing measurements of a person's blood, in accordance with some embodiments;

FIG. 2 is a block diagram illustrating components of a system for acquiring and processing spectroscopic data as a result of transdermal illumination with near infrared radiation, in accordance with some embodiments;

FIG. 3A is a diagram illustrating a handheld device that may be placed in contact with a person's forehead and that may be used to acquire and process measurements of a person's blood based on the reflection/backscatter of near infrared wavelengths by blood in the person's forehead and its subsequent detection, in accordance with some embodiments;

FIG. 3B is a diagram illustrating a wristband that may be used to acquire and process measurements of a person's blood based on the reflection/backscatter of near infrared wavelengths by blood in the person's arm and its subsequent detection, in accordance with some embodiments;

FIG. 3C is a diagram illustrating an apparatus in the form of a clip that may be placed over a person's finger and that may be used to acquire and process measurements of a person's blood based on the transmission and/or forward scatter of near infrared wavelengths through the person's finger and its subsequent detection, in accordance with some embodiments;

FIG. 3D is a diagram illustrating a clip that may be placed over a person's finger and that may be used to acquire and process measurements of a person's blood based on the reflection and/or backscatter from blood inside the persons' finger and its subsequent detection, in accordance with some embodiments;

FIGS. 3E1 and 3E2 show a measurement apparatus comprising a clip that may be attached to a person's ear and that may be used to acquire and process measurements of a person's blood based on transmission through the earlobe (FIG. 3E2) or reflection/backscatter from blood in the earlobe (FIG. 3E1), in accordance with some embodiments;

FIG. 3F is a diagram illustrating a clip that may be placed over a person's finger and that may be used to acquire and process measurements of a person's blood based on the transmission of near infrared wavelengths through the person's finger using a Fabry-Perot interferometer and its subsequent detection, in accordance with some embodiments;

FIG. 4 is a block diagram illustrating a system for acquiring and processing spectroscopic data as a result of the illumination of a person's finger by near infrared radiation, in which a person's finger is placed in between a source of illumination and a sensor or detector, in accordance with some embodiments;

FIG. 5A shows a measurement apparatus comprising a clip that may be placed over a person's finger and that may be used to acquire and process measurements of a person's blood, and that includes an actuator to vary the force applied to a persons' finger and a force sensor to determine the force applied during a measurement, in accordance with some embodiments;

FIG. 5B shows the apparatus of FIG. 5A in which a pressure regulator connected to a finger cuff is used to vary the flow of blood in a person's finger to assist in making a measurement, in accordance with some embodiments;

FIG. 5C shows a vacuum pump and vacuum chamber configured to alter a person's blood flow into a region as part of making a measurement, in accordance with some embodiments;

FIG. 5D shows a vacuum pump and vacuum chamber and other components as in FIG. 5C in which the source of illumination and the detector or sensor are oriented differently than in FIG. 5C, in accordance with some embodiments; and

FIG. 6 is a flowchart or flow diagram illustrating a process, method or operation for performing non-invasive spectroscopic measurements of a person's blood to determine a level, such as a value, amount or concentration, of a surrogate for a biomarker and using the change in that level or concentration over a time interval to generate a suggested change to the person's diet or other aspect of their lifestyle, in accordance with some embodiments.

### DETAILED DESCRIPTION

The following detailed description and provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

Although reference is made to measurements of a person, any suitable subject can be measured such as a person or animal with blood vessels.

Embodiments of the system, apparatuses, devices, and methods described herein are directed to improving a person's health by the measurement and evaluation of characteristics of a person's blood that serve as a surrogate for a biomarker. In some embodiments, a level of a surrogate for a biomarker is determined, in which the level comprises one or more of a value, an amount, or a concentration of the biomarker. In some embodiments, based on the change in the level of the surrogate over a period of time and its relationship to the expected variation in the value or level of the biomarker during that period, a recommendation may be generated to assist a person to improve their health. In some embodiments, measurements of the characteristic of the person's blood may be performed in a non-invasive manner, for example, by using near infrared spectroscopy. In some embodiments, the spectroscopy device may comprise a source of illumination, optical elements, a scanning mirror, and a sensor or detector, along with a controller. The controller may execute a set of computer-executable instructions that operate to control the output of the source of illumination, the motion of the scanning mirror and the processing of the signals that are sensed or detected.

In some embodiments, a spectroscope is used to collect data generated by interactions between light emitted by a source of illumination and fat molecules. In one example, this is performed by illuminating a persons' skin with near infrared light, where the source of illumination may be a light emitting diode or other light source and may be capable of varying the wavelength of the light it produces. By selecting a desired wavelength or wavelength band of the emitted light, the spectroscope may be used to detect fat molecules, which have well defined absorption properties at certain wavelengths and within certain wavelength bands. The near infrared light passes through the skin and is partially absorbed and/or scattered by fat molecules. The light received by a detector or other form of sensor may then be analyzed to obtain information about a level or concentration of fat molecules.

While fat molecules may be detected efficiently by this technique, there is still uncertainty as to the location of the fat molecules. This is because the illuminating light passes through skin, fat layers, and blood, all of which may contain fat molecules. However, the fat molecules in the blood may be identified because of a time varying component of the total signal received by the spectroscope. This is because as a person's heart pumps blood through their body, their veins carrying the blood undergo a slight expansion and contraction. This expansion and contraction cause a component of the signal(s) detected by the spectroscope to vary in a way that correlates to the beating of the heart. As a result, the pumping of blood by the heart introduces a correlated and time-varying component (termed a “pulsatile” component) to the fat absorption signals detected by the spectroscope that arise from interactions with the fat molecules in the blood. This means that the contribution of the fat molecules in the blood to the detected spectroscope signals may be distinguished from the contribution of fat molecules in the skin and fat layer by determining the component of the total fat molecule absorption signal that varies in time in a way that is strongly correlated with a person's heartbeat or pulse.

The correlation between a person's heartbeat or pulse and the time variation of a component of the spectroscope signal arising from interactions between fat molecules in the blood and the spectroscope's source of illumination provides a way to “measure” the level or concentration of fat molecules in a person's blood. This can be done by detecting a person's heartbeat/heartrate using a pulse oximeter, for example. By knowing the variation in blood flow as a function of time due to the beating of the heart, the correlated fluctuation in the signal arising from the interaction of the emitted light and fat molecules can be identified and allows a measurement of the level of fat in the blood over time. Further, because the level of fat in the blood varies (at least over timescales such as hours or days) primarily due to variations in the level of triglycerides in the blood, embodiments can measures changes in the level of fat in the blood over the course of several hours or during a day to infer that the level of triglycerides varied by a similar factor or percentage. Thus, in some embodiments, spectroscopic data regarding fat molecules is used together with information regarding a person's heartbeat or blood flow changes to provide an indication of the variation of fat molecules in the blood over a time interval, and that measurement is used as a surrogate for the variation of a biomarker (triglycerides) over the time interval.

In some embodiments, the spectroscopic measurements may be performed in a non-invasive and transdermal manner by the placement of a person's finger on a window or opening into a module containing some or all of the components of a spectroscope or placing such a module against a person's forehead. In other embodiments, the source of illumination may be in a separate module from the sensor or detector, and the measurements are made by placing a person's finger or earlobe in a space between the two modules. In some embodiments, measurements of the person's blood may be based on reflected or backscattered light from the person's finger, forehead, or earlobe. In other embodiments, measurements of the person's blood may be based on light transmitted through the person's finger or earlobe, with a sensor or detector positioned opposite to the source of illumination and the person's finger or earlobe positioned in between the source of illumination and the sensor or detector.

In some embodiments, a fat level in a person's blood is measured at two different times within a time interval and used as a surrogate for the change in the level of a biomarker over that time interval. In some embodiments, the biomarker is the level of triglycerides in the person's blood. In some embodiments, the change in the level of the biomarker maybe used to recommend or suggest that the person make a change to their diet, exercise regimen, or other aspect of their lifestyle in order to improve their health.

A number of biological constituents have absorption peaks in the near infrared (NIR) spectral region (700-2500 nm) and their impact on the spectrum of transmitted radiation can be determined by appropriate signal processing. This allows the identification of the constituents of a person's blood that interact with NIR radiation, such as by absorption or backscattering. Thus, by knowing the spectrum of transmitted radiation directed at a persons' blood and the received backscattered and/or transmitted spectrum after interaction with the constituents of the blood, those constituents and their level in the blood can be determined. This information can then be used to track the levels of those constituents over time; importantly, it can also be used to track the levels of biomarkers over time for which those constituents serve as surrogates.

NIR radiation has the advantage over other types of spectroscopic measurements in that body fluids and soft tissues are relatively transmissive at those wavelengths, e.g. translucent or substantially transparent. Thus, NIR radiation will pass through tissue and be absorbed or backscattered by various molecules in the blood. Processing of the signals resulting after the absorption or backscattering events can be used to identify the molecules responsible for the absorption of certain wavelengths or the interaction with the transmitted spectrum.

In some embodiments, NIR spectroscopy such as transdermal NIR spectroscopy is a relatively rapid analytical method that does not rely on sample preparation and reagents. By measuring the transmission, forward scatter, and/or reflection (such as backscatter) properties of a person's blood flow at different NIR wavelengths, the concentration of certain constituents can be determined. In some embodiments, these constituents include one or more of the fat level and protein. An aspect of NIR spectroscopy is that the NIR spectrum contains the required information about an investigated material (such as a person's blood), and concentrations of certain constituents in the material can be determined by the mathematical analysis of the spectra of reflected or transmitted radiation.

In some embodiments, transdermal NIR is used to determine an amount of a surrogate for triglyceride by measuring one or more of fat or protein. Examples of blood components that are detectable with fat using spectroscopic signals include blood components such as cholesterol and triglycerides. Work in relation to the present disclosure suggests that triglycerides levels in blood undergo an intraday variation whereas cholesterol levels remain substantially constant intraday as well as at least some non-triglyceride fat components of blood. Accordingly, in some embodiments, intraday variation in measured levels of fat in flood correspond to amounts of triglycerides in blood and can be correlated with triglyceride levels.

While the spectrometer system can be configured in many ways, in some embodiments, the spectrometer system is configured to lock onto a pulse signal of the person's blood to determine the level of fat in blood. The pulsatile fat signal provides changes that are indicative of the levels of fat in blood and can be distinguished from other types of non-circulating fat, such as cellular fat and adipose tissue.

The spectrometer can be configured to measure the spectra with any suitable wavelengths. In some embodiments, the spectrometer is configured to measure fat absorbance of light comprising a wavelength within a range from 1000 nanometers (nm) to 1100 nm. Alternatively or in combination, the spectrometer can be configured to measure one or more fat absorbance bands with wavelengths within a range from about 1400 nm to about 1600 nm, for example within a range from about 1450 nm to about 1550 nm, e.g. about 1500 nm. The detector to measure the light and corresponding fat absorbance may comprise any suitable detector, such as a silicon (Si) detector for measuring light wavelengths within the range from about 1000 nm to about 1100 nm, or an Indium Gallium Arsenide (InGaAs) detector for measuring longer wavelengths, e.g. within a range from 1400 to 1600 nm.

In some embodiments, the light source and detector are configured to measure spectral data at wavelengths within a range from about 1350 to about 1550 nm. Work in relation to the present disclosure suggests that blood pulsatile data from spectral measurements comprising wavelengths within a range from about 1350 to 1550 nm can be particularly well suited for measuring amounts of biomarkers and surrogate biomarkers such as one or more of fats, lipids, total lipids, triglycerides, cholesterol, total cholesterol, lipoprotein, high density lipoprotein, or low density lipoprotein, for example. The biomarkers and surrogate biomarkers can be measured with one or more of forward scatter, back scatter, transmission, or reflectance configurations of the one or more light sources and one or more detectors as disclosed herein.

In some embodiments, the spectrometer comprises a wavelength selective device such as a diffraction grating or an etalon to measure spectra. The wavelength selective device, e.g. etalon or grating, can be located on the transmission side of the spectrometer to filter light transmitted to the sample, or on the receive side to filter light transmitted from the sample to the detector. In some embodiments, the wavelength selective device, e.g. an etalon or diffraction grating, comprises a microelectromechanical (MEMs) filter. With an etalon, a distance between reflective surfaces of the etalon can be varied in order to filter the light transmitted through the etalon. With a grating, an angle of the grating or MEMs pixels can be varied in order to selectively transmit or receive wavelengths of light.

The spectral intensity signals can be measured and the wavelength selectively device varied in order to produce spectral intensity signals. In some embodiments, the spectral data is transformed with an appropriate transform function such as a Hadamard transform or a Fourier transform, in order to determine the spectra of the sample and associated peaks. Although reference is made to a detector, the detector may comprise an array of detector elements such as a linear one dimensional array or a two dimensional array.

In some embodiments wavelength selective device comprises a plurality of filters, such as an array of filters located in front of the plurality of detector elements.

In some embodiments, the spectrometer is configured to measure a plurality of wavelengths of light within a range from 1000 nm to about 1500 nm and to determine one or more of the partial pressure of oxygen (O₂), the pulsed partial pressure of O₂, carbon dioxide (CO₂), or fat. In some embodiments, the spectrometer comprises a processor configured with instructions to measure the partial pressure of oxygen (O₂), the pulsed partial pressure of O₂, carbon dioxide (CO₂), and fat. Although reference is made to measurement is made with NIR light, other wavelengths of light can be used alternatively or in combination.

The presently disclosed systems, methods, devices and apparatus are well suited for combination with prior approaches to measuring aspects of blood, such as pulsed oximetry, and one or more components of a commercially available pulsed oximeter can be combined with NIR measurements in accordance with the present disclosure, and data from the pulsation of blood combined with the NIR measurements to determine amounts of the surrogate biomarker.

The presently disclosed systems, methods, devices, and apparatus are well suited for combination with prior commercially available wearable smart sensors, such smart rings, smart watches and smart bands, such as the Fitbit Inspire, Samsung Galaxy Watch, Apple Watch, Fitbit Sense, and Fitbit Luxe, for example. The measurement of a biomarker such as a surrogate biomarker, e.g. triglycerides, can be combined with other measurements from the wearable device such as heart pulse rate, respiration, blood oxygen, electrocardiogram (ECG) measurements, and detection of conditions such as arrythmias. The systems, methods, devices, and apparatus disclosed herein may comprise one or more components of these smart wearable sensors, such as an internal processor, memory and communication circuitry, and can be configured to communicate with smart phones and remote servers such as Amazon Web Services (AWS). In some embodiments, the wearable NIR measurements as described herein are communicated similarly to a wearable smart sensor data such as data measured with an Apple Watch.

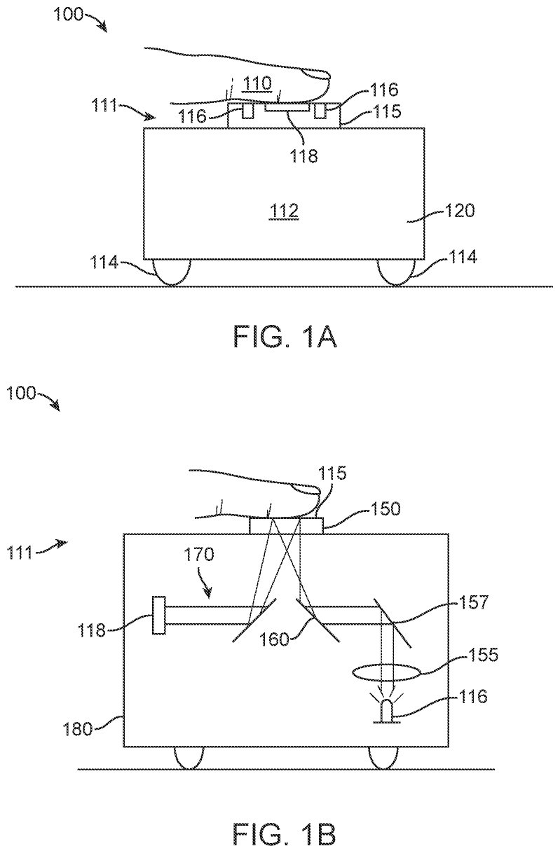

FIG. 1A is a diagram illustrating a persons' finger placed on a non-invasive apparatus 100 comprising a source of illumination, a sensor, and associated components of a near IR spectroscope for use in acquiring and processing measurements of a person's blood with back scatter. As shown in the figure, a person's finger 110 may be placed onto finger rest 115 of a module 112 in order to measure a characteristic of their blood using NIR spectroscopy. Module 112 may include supports 114, one or more illumination sources 116, a sensor or detector 118, and internal electronics 120. In some embodiments, the one or more illumination sources 116 and the detector 118 are arranged in a back scatter measurement configuration 111. The internal electronics may comprise a processor and can be configured to control the one or more illumination sources, obtain data from the sensor or detector and process that data to determine the spectral components of the data and/or the constituents of the person's blood.

FIG. 1B is a diagram illustrating a persons' finger 110 placed on a window 150 of a non-invasive apparatus 100 comprising one or more illumination sources, a sensor or detector 118 and associated components of a near IR spectroscope for use in acquiring and processing measurements of a person's blood. In some embodiments, the one or more illumination sources 116 and the detector 118 are arranged in a back scatter measurement configuration 111. As shown, a person's finger 110 may be placed on an optically transmissive window 150, e.g. a substantially transparent window, in order to permit the transmitted NIR radiation to reach their finger, pass through their skin and be absorbed and/or backscattered by constituents of their blood. In some embodiments, the optically transmissive window comprises the finger rest 115. The NIR radiation may be generated in a known spectrum by a source of illumination 116, which in some embodiments may be one or more light emitting diodes (LEDs) or a lamp, for example. The NIR radiation may be passed through one or more optical elements such as one or more lenses 155 to assist in forming a substantially collimated beam of light rays, which then are directed to a scanning mirror 160 by a diffraction grating 157 or other reflective element such as a mirror. The scanning mirror 160 can be configured in many ways and may comprises a digital mirror device (DMD) comprising a plurality of independently movable micro mirrors, a deformable mirror, or rotatable mirror such as one or more mirrors rotated with galvanometers. The scanning mirror 160 is operated to cause the NIR radiation to scan the person's skin, passing through their skin and interacting with the constituents of their blood. In some embodiments, the interactions are a combination of one or more of transmission, absorption, reflection, forward scattering, or backscattering. In some embodiments, the backscattered radiation passes back through the skin and the window to a diffraction grating 170 or other optical element which directs the radiation to the sensor or detector 118.

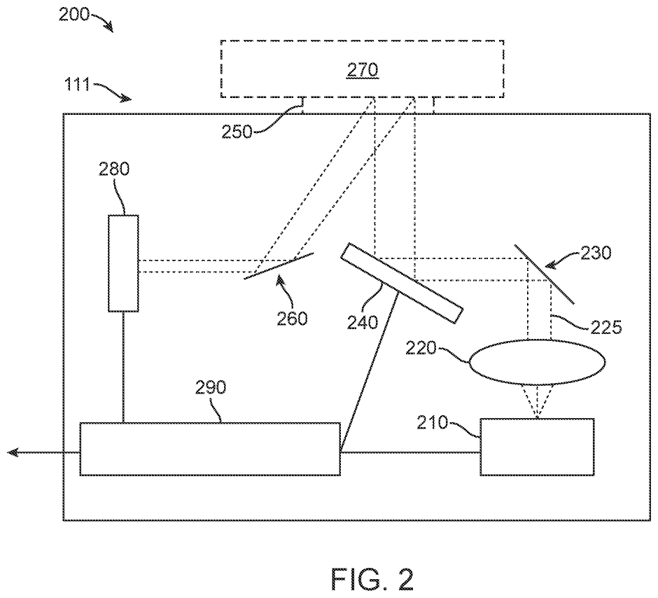

FIG. 2 is a block diagram illustrating components of a system for acquiring and processing spectroscopic data as a result of transdermal illumination, e.g. of a person's finger, by near infrared radiation, in accordance with some embodiments. As shown in the figure, in some embodiments, system 200 comprises one or more illumination sources 210, one or more optical elements 220, a mirror or diffraction grating 230 for directing (and in some cases selecting) wavelengths of radiation generated by source 210, a scanning mirror 240, a window 250, a mirror or diffraction grating 260 for directing radiation received from a sample (such as a person's finger 270), a sensor or detector 280, and a controller or processing element 290. The components of system 200 may comprise one or more components of system 100, and vice versa. In some embodiments, the one or more illumination sources 210 and the detector 280 are arranged in a back scatter measurement configuration 111.

Source 210 may comprise a single element or multiple elements. In some embodiments, source 210 comprises one or more light emitting diodes (LEDs) that operate to generate wavelengths of light in the near infrared region of the spectrum (although they may also generate light in other wavelengths). Optical elements 220 may comprise one or more lenses, partial mirrors, etc. that operate to produce a substantially collimated beam 225 from the light generated or emitted by source(s) 210. Mirror or diffraction grating 230 functions to redirect the beam 225 produced by optical elements 220 to a scanning mirror 240. If a diffraction grating is used it may function to selectively filter certain wavelengths of light based on the spacing of the grating elements, for example. This may result in removing certain wavelengths of light produced by source 210 from the beam provided to scanning mirror 240. Scanning mirror 240 may be operated to move the beam 225 received from mirror/grating 230 in such a way that it passes through window 250 (which is transmissive to near IR wavelengths) and illuminates a person's tissue, e.g. finger 270, for example. The near IR radiation passes through the skin and interacts with molecules in the person's blood. The interactions may include backscattering of radiation back through window 250. The backscattered radiation is redirected by mirror or grating 260 to a sensor or detector 280. Depending upon the wavelengths being used to determine the level of the biomarker surrogate, sensor 280 may be of Silicon, InGaAs, or another suitable material. Sensor or detector 280 provides signals to controller 290. In some embodiments, controller 290 may be an electronic processor that is programmed to execute a set or sets of computer-executable instructions. The instructions may configure controller 290 to perform one or more functions, operations, or methods as described.

As described, elements of the NIR spectroscope or spectroscopy system 200 may include a controller 290 that comprises a processor programmed with a set of computer-executable instructions. When executed, the instructions may cause the controller to perform one or more of (a) control the spectrum of radiation produced by the source of illumination 210, including varying the radiation over certain wavelength bands, control the movement of the scanning mirror 260, receive and process signals generated by the sensor or detector 280 in response to sensing or detection of the backscattered radiation from the person's blood, and based on spectroscopic analysis of the received signals, process the received signals to determine a concentration or level of a constituent of the blood and/or provide the received signals to an external processing element for performing the spectroscopic analysis and subsequent processing.

Further, although not shown in the figure, a system that includes the spectroscopic device of FIG. 2 may also include an electronic processor that executes a set of computer-executable instructions in order to perform one or more functions, operations, or methods. In some embodiments, the measurement apparatus is connected to a mobile electronic device such as a smart phone configured to receive measurement data, and the measurement data can be stored. These functions, operations, or methods may include, but are not limited to or required to include (a) determining the concentration or level of the constituent of the blood at different times during a time interval, (b) determining a change in the level or concentration of the constituent during the time interval, (c) inferring or determining the corresponding change in the concentration or level of a biomarker in the blood during the time interval, and (d) based on the changed level or concentration (or in some cases, the level) of the biomarker in the blood during the time interval, generate a recommendation or suggestion to the person to do one or more of change or experiment with a change in their diet, change or experiment with a change in their exercise regimen and change or experiment with a change in another aspect of their lifestyle.

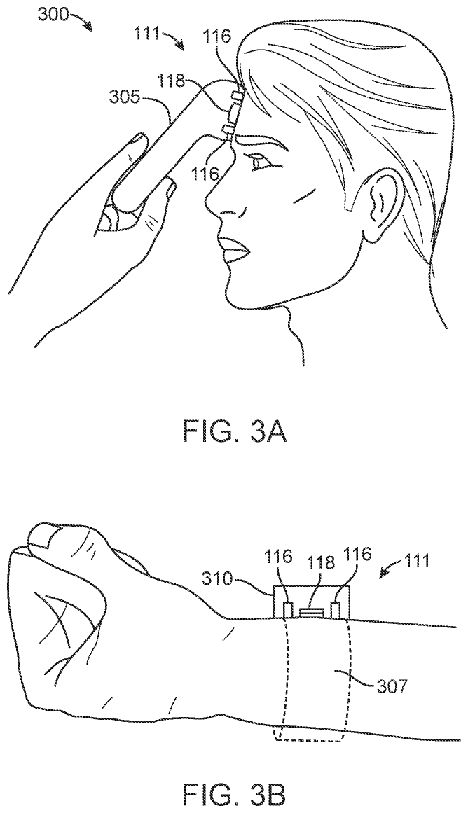

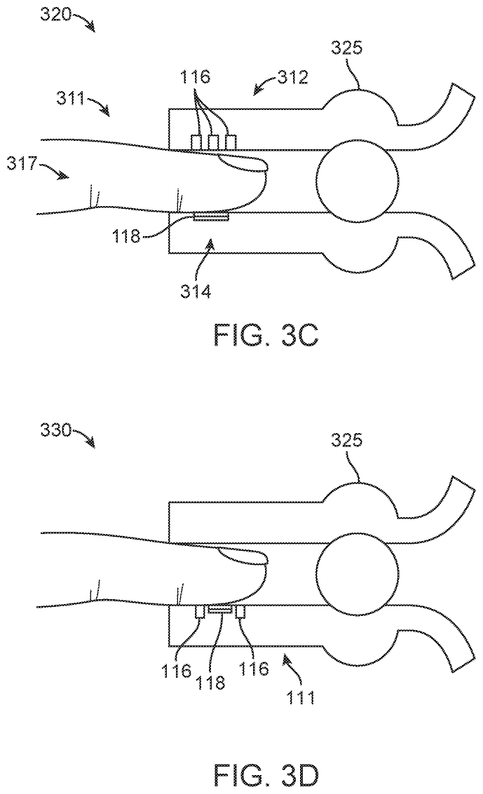

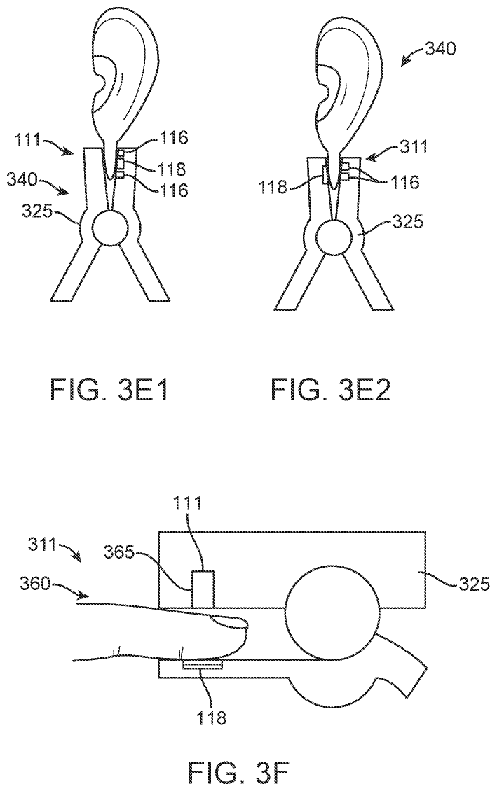

Although FIGS. 1A, 1B and 2 illustrate the components or elements of the spectroscopy device as a module upon which a person's finger can be placed, in some embodiments, the device may be incorporated into other forms or form factors. As examples, in some embodiments the modules illustrated in those figures may be incorporated into a handheld device that may be placed against a person's forehead in order to measure their blood, as shown by FIG. 3A. In some embodiments, the apparatus 300 is used to acquire and process measurements of a person's blood based on one or more of the reflection or backscatter of near infrared wavelengths by blood in the person's forehead and its subsequent detection. The apparatus 300 may comprise a handle 305 to position the one or more illumination sources 118 and detector or sensor 118 in proximity to the forehead of the person. In some embodiments, the one or more illumination sources 116 and the detector 118 are arranged in a back scatter measurement configuration 111.

In another example, the module may be incorporated into a wrist worn apparatus 310 comprising a band 307, in order to obtain measurements by being placed against a person's wrist or arm, as shown by FIG. 3B. In some embodiments, the device is used to acquire and process measurements of a person's blood based on one or more of the reflection or backscatter of near infrared wavelengths by blood in the person's arm and its subsequent detection. In some embodiments, the one or more illumination sources 116 and the detector 118 are arranged in a back scatter measurement configuration 111. The wrist worn apparatus 310 may comprise one or more components of a wearable smart device, such as a smart watch as described herein.

FIG. 3C is a diagram illustrating an apparatus 320 in the form of a clip 325 that may be placed over a person's finger and that may be used to acquire and process measurements of a person's blood based on the transmission and/or forward scatter of near infrared wavelengths through the person's finger and its subsequent detection. With transmission measurements, the one or more light sources 116 and the detector 118 are arranged on opposite sides of the tissue with a transmission configuration 311.

The measurement apparatus may comprise a light source module 312 separated from the sensor or detector module 314 with a space 317 in between to receive tissue to measure the surrogate biomarker with an optical transmission measurement configuration. The light source module 312 may comprise the one or more illumination sources 116 and described herein, and the detector module 314 may comprise the sensor or detector 118 as described herein. The apparatus can be configured in many ways and can be configured to transmit light energy through a first portion of the skin on a first side of the received tissue and to receive light from a second portion of the skin on a second side of the received tissue. In some embodiments, the measurement apparatus is configured to receive tissue such as a finger, earlobe, or fold of skin, into the space, for example to permit a person's finger to be inserted into the space. The tissue received in the space can be measured with either the transmission configuration 311, or with the backscatter configuration 111.

FIGS. 3C, 3D, 3E and 3F show clip embodiments, in which the measurement apparatus comprising the one or more illumination sources 116 and sensor or detector 118 are supported on the skin with clip 325.

In some embodiments, each of the two parts of a clip may incorporate a module with tissue such as a person's finger, fold of skin or earlobe placed in between for transmission measurements, as shown by FIGS. 3C, 3E (right side) and 3F. Alternatively, the clip can be configured to measure the surrogate biomarker with back scatter as shown in FIGS. 3D and 3E (left side).

FIG. 3D shows a measurement apparatus 330 comprising clip 325 that may be placed over a person's finger and that may be used to acquire and process measurements of a person's blood based on the reflection and/or backscatter from blood inside the persons' finger and its subsequent detection. The clip 325 may comprise the one or more illumination sources 116 and detector 118 arranged in a backscatter measurement configuration 111.

FIGS. 3E1 and 3E2 show a measurement apparatus 340 comprising a clip 325 that may be attached to a person's ear. The apparatus 340 may comprise one or more illumination sources 316 and detector 118 arranged in either a back scatter measurement configuration 111 as shown in FIG. 3E2, or a transmission measurement configuration 311 as shown in FIG. 3E1. The apparatus 340 comprising the clip 325 may be used to acquire and process measurements of a person's blood based on transmission through the earlobe or reflection/backscatter from blood in the earlobe.

FIG. 3F show a measurement apparatus 360 comprising a clip 325 that may be placed over a person's finger and that may be used to acquire and process measurements of a person's blood based on the transmission of near infrared wavelengths through the person's finger using a Fabry-Perot interferometer and its subsequent detection. The Fabry-Perot interferometer can be configured in many ways and may comprise a variable spacing between reflective surfaces in order to sweep measurement wavelengths from light source 111. Alternatively or in combination, the Fabry-Perot interferometer may comprise an etalon configured to pass discrete wavelengths of light, as will be understood by one or ordinary skill in the art. Although the Fabry-Perot interferometer is shown with the transmission measurement configuration, the interferometer can be configured in the backscatter measurement configuration as described herein.

In some embodiments, the source of illumination is positioned on the same side of a person's skin as the sensor or detector in configuration 111, and backscatter or reflected radiation is measured and analyzed. In alternative embodiments the source of illumination is positioned on an opposite side of a person's skin as the sensor or detector, and transmitted radiation is measured and analyzed with configuration 311.

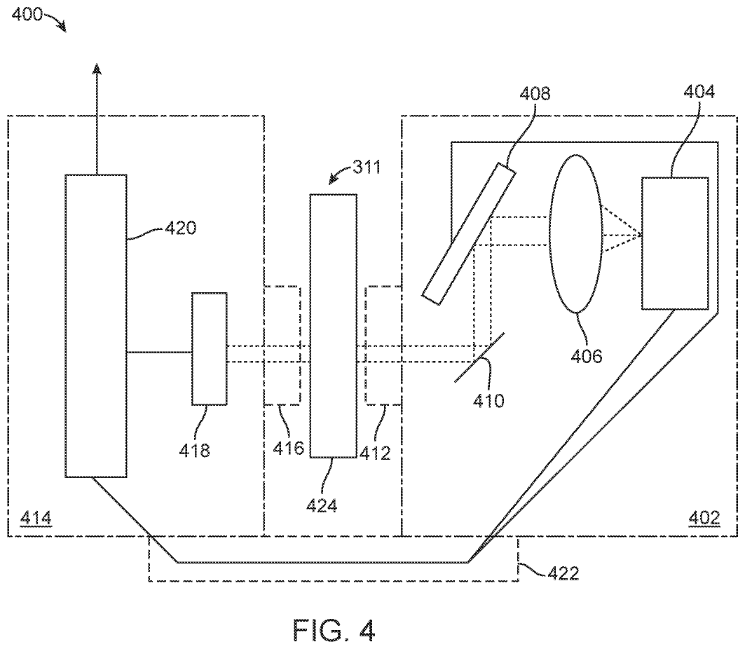

FIG. 4 shows components of a system 400 for acquiring and processing spectroscopic data as a result of the transmission illumination of a person's finger by near infrared radiation. In some embodiments, a person's finger, fold or skin or earlobe is placed in between a source of illumination and a sensor or detector in a transmission configuration 311. In some embodiments, system 400 may comprise two primary modules that are positioned to allow a person's finger (or earlobe in some embodiments) to be placed in between the modules. A first module 402 may comprise one or more illumination sources 404, one or more optical elements 406, a scanning mirror 408, a mirror or diffraction grating 410, and a window 412. A second module 414 may comprise a window 416, a sensor or detector 418 and a controller 420 comprising a processor. Module 402 and module 414 may be connected by a channel 422 which contains electrical connections in addition to providing a support structure for the modules. Controller 420 may be configured to control the operation of the one or more sources 404, scanning mirror 408 and to receive signals sensed or detected by sensor 418. Further, controller may be configured to process those signals to spectroscopically analyze the received signals. In some embodiments, the spectroscopic analysis may occur in a separate component or element.

As shown in the figure, a person's finger may be inserted or placed between modules 402 and 414. Light from source 402 passes through window 412 and illuminates finger 424. The near IR radiation passes through the skin of finger 424 and interacts with molecules in the blood. The molecules absorb certain wavelengths, with the remaining wavelengths being transmitted through finger 424 to window 416. Based on a spectroscopic analysis of the transmitted wavelengths, a level or concentration of certain molecules may be determined. In some embodiments, these determined levels or concentrations may be that of a surrogate for a biomarker, for example, a level of fat molecules in the blood.

One or more components of system 400 can be combined with one or more components of embodiments comprising transmission configuration 311, and vice versa, for example with reference to FIGS. 3C, 3E2 and 3F.

The inventors have recognized that for non-invasive blood sensing, it may be beneficial to perform measurements in states representing different levels of blood volume in a person's veins and arteries. In some embodiments, the measurements described herein may be performed at both a relatively higher blood volume level and at a relatively lower blood volume level. A benefit of this sequential scanning or measuring process is that the difference in the measurements between the two states is expected to contain information about the person's blood that decreases the effect of the illumination of the skin and underlying tissue, for example with a difference measurement between the higher volume and the lower volume. This may provide more accurate data, as well as data that may be more useful for modeling and other purposes.

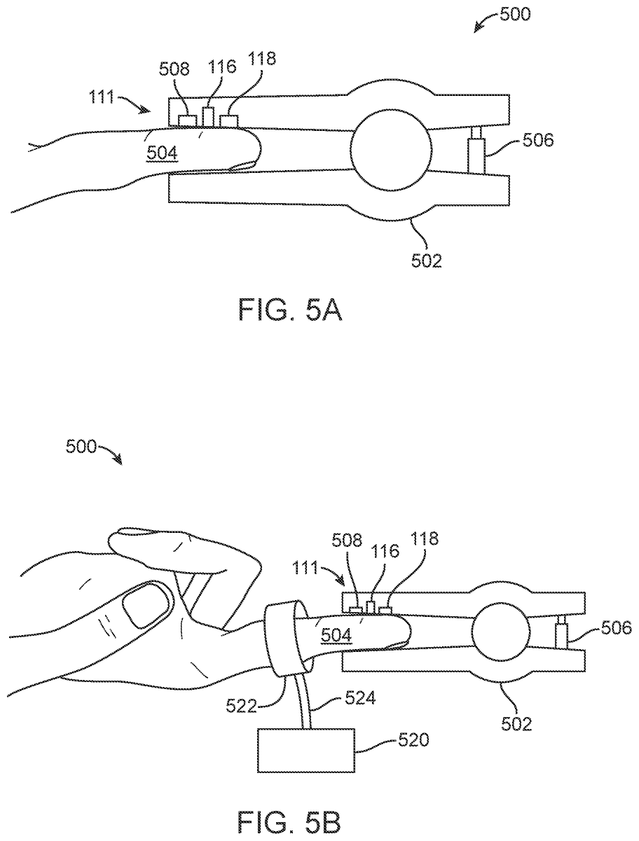

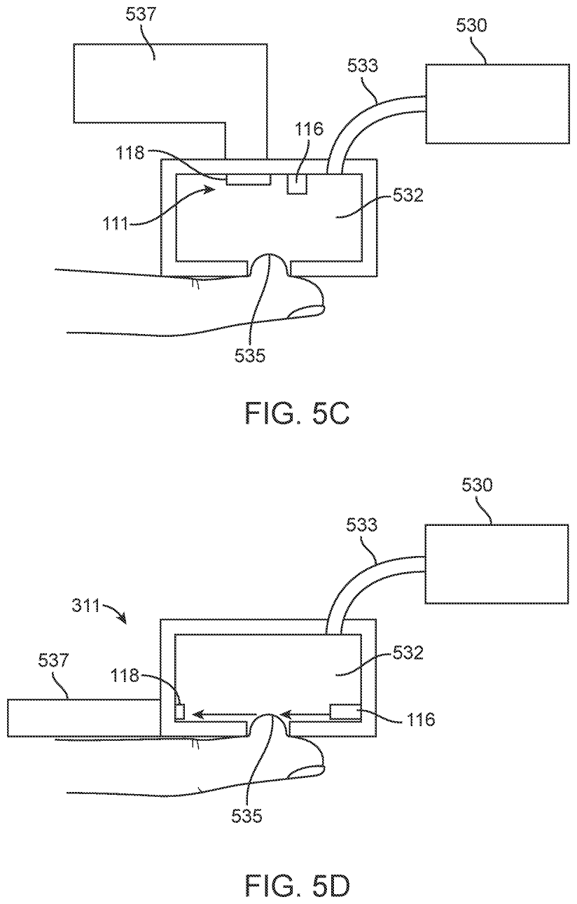

In some embodiments, the scan or measurement may be performed at both a high and a low point of the blood pulse or pulsatile component of the blood flow. In other embodiments, as are described with reference to FIGS. 5A-5D, an artificial source of pressure may be used to alter the volume of blood present in a person's veins or arteries during a measurement process.

FIG. 5A shows a measurement apparatus 500 comprising a clip 502 that may be placed over a person's finger 504 and that may be used to acquire and process measurements of a person's blood, and that includes an actuator 506 to vary the force applied to a persons' finger and a force sensor 508 to determine the force applied during a measurement. The measurement apparatus 500 may comprise the one or more light sources 116 and detector 118 arranged in the backscatter configuration 111. Alternatively, the one or more light sources and detector can be arranged in the transmission configuration. The actuator 506 is used to control the amount of force applied to a persons' finger. The actuator may be controlled by a direct or wireless connection to a controller as described herein, where the controller may be programmed to execute a set of instructions that determine the amount of force to be applied and to coordinate the applied force with the measurement process. A force sensor 508 may be used to monitor the force applied to the person's finger and may provide data that is used by the controller as part of the measurement process. Varying the force applied by controlling the actuator alters the blood volume in the affected blood vessels by either causing blood to move away from the area where the force is applied, or allowing blood to move into the tissue, e.g. by application of less or no force.

FIG. 5B shows the apparatus 500 of FIG. 5A in which a pressure regulator 520 connected to a finger cuff 522 is used to vary the flow of blood in a person's finger to assist in making a measurement. In this embodiment, an additional source of pressure is used to provide control over the blood flow in a person's finger and to assist in making more accurate measurements. As shown in the figure, in this embodiment, a cuff 522 is placed around the finger and the pressure applied to the finger by the cuff is controlled by a pressure regulator 520 connected to an air hose 524. The finger cuff 522 may be used to reduce the arterial flow of blood to the finger momentarily, thereby reducing the blood volume in the finger during a scan or measurement.

FIG. 5C shows a measurement apparatus comprising a vacuum pump 530 and vacuum chamber 532 configured to alter a person's blood flow into a region as part of making a measurement. In some embodiments, a vacuum chamber 532 is placed over a region of a person's finger and a vacuum pump 530 coupled to a hose 533 is used to reduce the pressure within the chamber. This may be used to cause the person's skin to bulge as blood is forced into a region of the finger by the difference in pressure between the portion of the finger inside the vacuum chamber and the portion outside of the chamber. Varying the degree of vacuum in the chamber may be used to cause the blood in the affected region to vary between states of higher and lower volume. The one or more light sources 116 and detector 118 can be arranged in a back scatter configuration 111. In some embodiments, the detector 118 comprises one or more optics and sensors 537 as described herein.

FIG. 5D shows a measurement apparatus comprising a vacuum pump 530 and vacuum chamber 532 and other components as in FIG. 5C in which the source of illumination and the detector or sensor are oriented differently than in FIG. 5C. The one or more light sources 116 and the detector 118 may comprise the transmission configuration 311. This embodiment may be used to obtain a side-view scan. The side view scan may rely on a combination of optical transmittance and forward scatter of light. Work in relation to the present disclosure suggests that this approach may include less scatter and more absorbance from the person's tissue than embodiments relying on backscattered light as described herein.

Although FIGS. 1A, 1B, 2, 3A, 3B, 3C, 3D, 3E1, 3E2, 4, 5A, 5B, 5C, or 5D show systems, devices, and apparatus for measuring surrogation biomarkers in accordance with some embodiments, one of ordinary skill in the art will recognize many adaptations and variations. In some embodiments, the one or more light sources comprises a plurality of light sources configured to measure pulsatile blood data, e.g. pulsed oximetry data, with a first light source having a first wavelength shorter than an isobestic point of oxyhemoglobin and deoxyhemoglobin, e.g. at about 805 nm +/−5 nm, and a second light source having a second wavelength longer than the isobestic point of oxygen. This pulsatile data such as pulsed oximetry data can be combined with the NIR measurements of surrogate biomarkers as disclosed herein, in order to determine blood components of the one or more surrogate biomarkers. The first light source may comprise a first one or more wavelengths within a range from about 600 nm to about 700 nm, for example within a range from about 630 nm to about 690 nm, and the second light source may comprise a second one or more wavelengths within a range from about 900 nm to about 1000 nm, for example within a range from about 910 nm to about 970 nm. The one or more detectors can be configured to measure the light from the one or more sources after passing through tissue as described herein. In some embodiments, pulsed oximetry data is output similarly to conventional pulsed oximeters, e.g. one or more of partial oxygenation, pulse rate, and respiration rate, in addition to surrogate biomarker data.

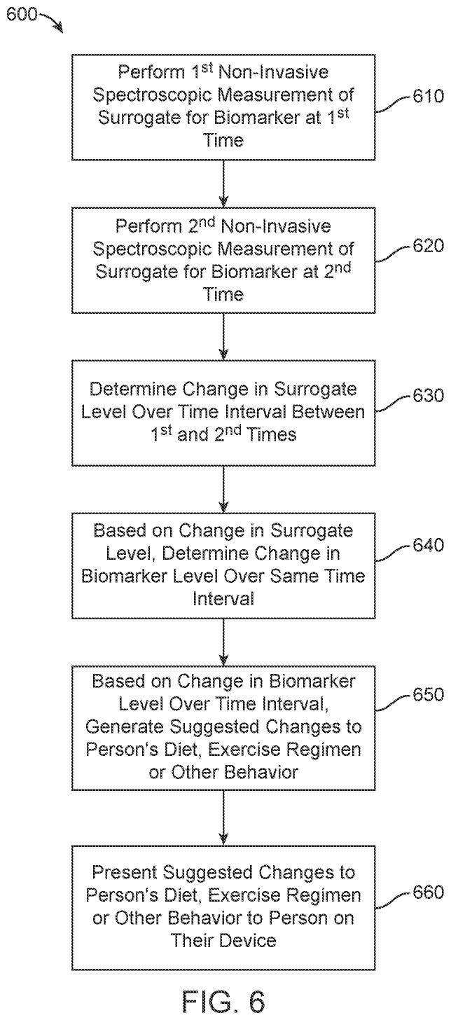

FIG. 6 is a flowchart or flow diagram illustrating a process, method, or operation 600 for performing non-invasive spectroscopic measurements of a person's blood to determine a level or concentration of a surrogate for a biomarker and using the change in that level or concentration over a time interval to generate a suggested change to the person's diet or other aspect of their lifestyle, in accordance with some embodiments. As shown in the figure, at step or stage 610, a first non-invasive spectroscopic measurement of a surrogate for a biomarker in a person's blood is performed at a first time. The measurement (and in some cases spectroscopic analysis) may performed by a system or device as shown in FIGS. 1A, 1B, 2, 3A, 3B, 3C, 3D, 3E1, 3E2, 4, 5A, 5B, 5C, or 5D as described herein.

At a later and second time (as suggested at step or stage 620), a second non-invasive spectroscopic measurement of the surrogate for the biomarker in the person's blood is performed. A processing element or controller then determines the change in the level or concentration of the surrogate for the biomarker during the time interval between the first and second time, as suggested by step or stage 630. At step or stage 640, the change in the level or concentration of the surrogate is used to determine the change in the level or concentration of the biomarker during that time interval. At step or stage 650 and based on the change in the level or concentration of the biomarker, a suggested or recommended change to a person's diet, exercise regimen, or other aspect of their lifestyle or behavior may be generated. The suggestion or recommendation is then presented to the person as a user interface or notification on their device (as suggested by step or stage 660).

In some embodiments, the suggestion or recommendation may comprise one or more of (a) an alert that the person's biomarker levels have increased or decreased significantly during the time interval, (b) a suggestion or recommendation to participate in a specific experiment to determine if the fluctuation in the person's′ biomarker level can be altered, such as by changing the person's diet or exercise regimen, and (c) a suggestion or recommendation to perform another measurement of the person's blood at a later time.

Although reference is made to a scanning digital mirror, the presently disclosed methods and apparatus can be combined with other types of spectroscopy such as Fourier Transform Infrared (FTIR) spectroscopy, and dispersive spectrometers. By way of example, the presently disclosed spectrometer or spectroscopic analysis element may comprise one or more components of the commercially available DLP NIRSCAN Evaluation Module, commercially available from Texas Instruments.

The spectrometer can be configured in different ways, and may comprise one or more components of known spectrometers, such as a Fourier Transform Infrared (FTIR) spectrometer, a dispersive spectrometer with a detector array, or a spectrometer with a tunable laser as described in U.S. application Ser. No. 14/992,945, filed on Jan. 11, 2016, entitled “Spectroscopic measurements with parallel array detector”, published as US20160123869A1 on May 5, 2016, the entire disclosure of which is incorporated herein by reference. In some embodiments the spectrometer comprises a tunable laser as a source of illumination, for example.

In some embodiments, processing of the sensed or detected signals may occur externally to the spectroscope device or apparatus used to perform the measurements and collect the data. For example, a computing device may be communicatively coupled to the spectroscope or other device used to acquire the blood measurement data and to a network. The computing device may be used to perform analysis of the wavelength spectra and/or other data obtained from measurement(s) of the person's blood, where the data is obtained from the spectrometer or other measurement device. In this regard, the computing device may be in communication with the network element to retrieve information pertaining to blood analysis such that a user of the computing device (e.g., a medical professional, a trainer, or the like) can analyze the wavelength spectra from the spectrometer and provide a diagnosis and/or other relevant information pertaining to the user/patient of the spectrometer. Examples of the computing device include computers, smart phones, and the like, comprising various hardware, software, and/or firmware components for processing the wavelength spectra from the spectrometer.

In some embodiments where the spectrometer is communicatively coupled to a network, the spectrometer may be able to communicate wavelength spectra to a computing device. For example, the computing device may also include computers, smart phones, and the like, comprising various hardware, software, and/or firmware components for processing the wavelength spectra and/or other spectral data from the spectrometer.

The spectrometer, the computing devices, and the network element, either alone or in combination, may be configured with instructions (e.g., software components) that direct a processor to perform one or more types of data analyses. For example, a processor configured with the spectrometer, the computing devices, and/or the network element may determine a concentration or level of a surrogate for a biomarker (such as the fat level in the blood as described herein). In some embodiments, the processor is configured with instructions to measure the sample at a plurality of times.

In some embodiments, a processor can be configured with instructions to enable a user to conduct an experiment with a plurality of blood measurements from the user (or another subject). For example, the processor can be configured for the user to select one or more experiments, such as one or more of a metabolism experiment, a cardiovascular health experiment, or an inflammation and immune function experiment. In some embodiments, a computing device of the system may comprise a recommendation engine that presents one or more lifestyle change experiments to a user via a graphical user interface of a user device, such as the user's computing device.

In response to the user selecting an experiment, the processor provides appropriate prompts for the user to conduct the experiment. The processor may comprise instructions to present an appropriate instruction to the user to conduct the experiment. For each type of experiment the processor can be configured with instructions to measure one or more of the surrogates or markers as described herein.

In some embodiments, the spectrometer comprises a broad spectrum light source to generate a plurality of wavelengths of light, a detector, and a wavelength selector coupled to the broad spectrum light source to selectively direct light toward the detector with the sample (e.g., the person's finger or earlobe) located between the wavelength selector and the detector. The wavelength selector may comprise one or more of a dispersive element, a prism, a diffraction grating, a digital mirror device (“DMD”), a diffractive optic, an interferometer, a Michelson interferometer, or an Etalon. In some embodiments, the spectrometer comprises a digital micromechanical mirror optically coupled to the wavelength selector to selectively reflect the light from the wavelength selector to the detector. In some embodiments, the detector comprises an indium gallium arsenide (InGaAs) detector. In some embodiments, the detector comprises a single element detector, while in other embodiments the detector comprises a plurality of detector elements.

As described, the system and methods disclosed herein may be used to measure a surrogate for a biomarker in a person's blood and use information regarding the level or variance of the surrogate to recommend changes to the person's nutrition, exercise regimen, or lifestyle. In some embodiments, the measurement(s) of the surrogate for the biomarker may be used to provide feedback to a person regarding an experiment they are conducting to improve their health.

In some embodiments, a surrogate for a biomarker refers to a measurable quantity that is correlated to a biomarker. For example, in some embodiments, the surrogate comprises the level of fat in a person's blood or blood plasma, and the biomarker to which it is correlated is a triglyceride level. Thus, in some embodiments, by making spectroscopic measurements of a person's blood sample at different times, the fat level in their blood may be determined at those times and the variance in fat level may be determined during a time interval. Based on the variance in the fat level, the variance in the person's triglyceride level during the time interval is inferred. Because the variance, and in some cases the level of triglycerides in a person's blood can be an indicator of health, the fat levels in a person's blood at various times or with reference to various activities (such as eating or exercising) can provide information that can be used to suggest ways for the person to improve their health.

Research performed in connection with the development of the system and methods described herein has indicated that the variance in the level of fat in a person's blood is strongly correlated with changes in the triglyceride level in the person's blood. This allows a spectroscopic measurement of the fat level in blood to be used as a surrogate or substitute for measuring triglyceride. Because of the significance of triglyceride levels and their variation to health (e.g., as a marker for inflammation due to diet, stress, and other factors that can impact health), the measurement of fat levels can provide assistance to a person as they attempt to modify their diet or exercise regimen to improve their health.

As an example, the system and methods described may be used to monitor a spike in triglyceride levels (as inferred from measurements of the surrogate fat level) as a result of eating or eating specific foods. This may be helpful to recommending dietary changes or lifestyle changes to a person so as to avoid or reduce such a spike. Further, monitoring fat levels over a time interval may provide data that can be used to develop a model of the person's response to fasting, diet, exercise, or other behaviors. The model may then be used to generate recommendations of activities to participate in, not participate in, or delay in order to minimize the negative impact of an elevated triglyceride level.

Still further, the reliability of the detection of a possible triglyceride spike may be impacted by how recently a person has eaten. In order to prevent this problem, in some embodiments, a measured level of glucose or protein, or an elevated level of glucose or protein may be used to advise a person to provide a blood sample at a different time.

In some embodiments, information regarding a person's activities in a time interval prior to their providing a blood sample for analysis may be collected and used to associate changes in their diet, consumption of a specific foods, or engaging in a specific activity with levels of a biomarker surrogate in their blood. This may allow a model to better characterize the person's physiology and improve recommended changes or “experiments” for that person. Further, collecting such data for a group of people may provide ways to compare a person's measurement(s) to that of a similarly situated group and thereby improve recommendations for the person.

In some embodiments, the system and methods may be configured to generate an alert if the level of a biomarker surrogate exceeds a threshold value or undergoes a significant variance during a time interval. This can inform a user of an association between their behavior or activity and the level or change in level of the measured surrogate.

While the example of the fat level in blood serving as a surrogate for triglycerides has been discussed, the system and methods described herein may be used to monitor other surrogates as well and other combinations of surrogates and correlated biomarkers. In some embodiments, a model of spectral data is used to determine an amount of a surrogate for a related biomarker or combination of surrogates and biomarkers. In some embodiments, a plurality of spectral signals is combined to determine an amount of a surrogate biomarker or biomarker. Examples of combinations of spectral signals to determine a biomarker which can be modified to determine an amount of a surrogate for a biomarker with transdermal measurements in accordance with the present disclosure are described in PCT/US2019/030052, filed on, entitled “SYSTEMS AND METHODS FOR BLOOD ANALYSIS”, published as WO2019213166 on Nov. 7, 2019, the entire disclosure of which is incorporated herein by reference. Examples of laboratory NIR measurements and chemometric analyses of lipids suitable for incorporation in accordance with embodiments the present disclosure are described in G. Bazar et al., “Multicomponent blood lipid analysis by means of near infrared spectroscopy, in geese”, Talanta 155 (2016) 202-211. The amounts of biomarker and surrogate biomarker can be determined in many ways in accordance with the present disclosure, for example with one or more of a genetic algorithm or principal component analysis, for example.

Based on the teachings provided herein, one of ordinary skill in the art can develop models to detect components of blood that are surrogates for one or more of albumin, globulin, fibrinogen, omega fatty acids, or additional biomarkers. Fibrinogen, for example, may correspond to increased inflammation and may correspond to another marker of inflammation such as C-reactive protein. Based on the teachings provided herein, one of ordinary skill in the art can conduct experiments to determine the sensitivity and specificity of instrumentation for surrogate biomarker detection and biomarker detection, such as the spectroscopy, circuitry, optical components and acquisition times, so as to provide suitable measurements in accordance with the present disclosure. Work in relation to the present disclosure suggests that there may be combined biomarkers or direct-from-spectrum, high-frequency biomarkers that can be identified with high frequency data, e.g. measured twice daily. In some embodiments, the high frequency blood data is matched with health outcomes, in order to build a predictive model of these outcomes. In some embodiments, the lifestyle actions are identified that tend to alter the levels of one or more of a surrogate biomarker, combinations of spectral channels corresponding to a biomarker, an individual chemical, or combined markers.

Although reference is made to changes in fat as a surrogate for triglycerides, the presently disclosed methods and apparatus can be used to determine surrogates for other biomarkers. The spectroscopic data referred to herein can be analyzed, e.g. mined, with one or more of deep learning, machine learning, convolutional neural networks or artificial intelligence to determine suitable spectral ranges and combinations of spectral ranges corresponding to surrogates for biomarkers. Spectral data can be acquired from several blood measurements and correlations determined for changes in surrogates and corresponding biomarkers. These surrogates corresponding to changes in biomarkers can be used to present data and suggested health experiments to a user as described herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively, or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, a “level” of a biomarker or surrogate encompasses one or more of a value of the biomarker or surrogate, an amount of the biomarker or surrogate, or a concentration of the biomarker or surrogate.

As used herein, characters such as numerals refer to like elements.

As used herein, the term “light” refers to electromagnetic radiation, which may comprise visible electromagnetic energy, near infrared electromagnetic energy, infrared electromagnetic energy, or ultraviolet electromagnetic energy.

The present disclosure includes the following numbered clauses.

Clause 1. A method, comprising: performing a first non-invasive measurement of a surrogate for a biomarker in a subject's blood at a first time; performing a second non-invasive measurement of the surrogate for the biomarker in the subject's blood at a second time; determining a change in the measurement of the surrogate for the biomarker during a time interval defined by the first time and the second time; and based on the determined change in the measurement of the surrogate for the biomarker, determining a change in the biomarker during the time interval.

Clause 2. The method of clause 1, wherein performing the first non-invasive measurement of the surrogate comprises performing a measurement of a signal that comprises a contribution from the surrogate and the method further comprises determining a component of the measured signal that varies in time in a manner that is correlated with the subject's heartbeat.

Clause 3. The method of clause 2, wherein performing the second non-invasive measurement of the surrogate comprises performing a measurement of a signal that includes a contribution from the surrogate and the method further comprises determining a component of the measured signal that varies in time in a manner that is correlated with the subject's heartbeat.

Clause 4. The method of clause 2, wherein the measurement of the signal comprises a measurement of fat molecules, and the measurement of the surrogate comprises a measurement of the fat molecules in the blood.

Clause 5. The method of clause 4, wherein the biomarker comprises one or more triglycerides.

Clause 6. The method of clause 1, wherein performing the non-invasive measurement comprises collecting spectroscopic data that includes a contribution from the surrogate for the biomarker.

Clause 7. The method of clause 6, further comprising using a near infrared spectroscopy device to collect the spectroscopic data.

Clause 8. The method of clause 7, wherein the near infrared spectroscopy device comprises a source of illumination, a sensor or detector, and a controller configured to operate the source of illumination and to receive signals from the sensor or detector.

Clause 9. The method of clause 1, wherein the non-invasive measurement comprises a transdermal measurement.

Clause 10. The method of clause 9, wherein performing the non-invasive measurement further comprises: configuring a window that is substantially transparent to near infrared radiation to enable its placement against the skin of the subject; operating a source of illumination to generate near infrared radiation; directing the generated radiation to pass through the window and intersect the skin of the subject; and measuring a component of the generated radiation after the radiation passes from the subject's skin back through the window.

Clause 11. The method of clause 10, wherein the window is configured to be placed against the subject's finger, arm, or forehead, and further, wherein the source of illumination and the sensor or detector are arranged to be on the same side of the subject's finger, arm, or forehead.

Clause 12. The method of clause 9, wherein performing the non-invasive measurement further comprises: configuring a first window that is substantially transparent to near infrared radiation to enable its placement against the skin of the subject; operating a source of illumination to generate near infrared radiation; directing the generated radiation to pass through the first window and intersect the skin of the subject; and measuring a component of the generated radiation after the radiation passes from the subject's skin through a second window.

Clause 13. The method of clause 12, wherein the first and second windows are configured to be placed on opposite sides of the subject's finger or earlobe.

Clause 14. The method of clause 1, further comprising presenting one or more of the following on a user interface of a device: an alert that the subject's biomarker levels have increased or decreased during the time interval; a suggestion or recommendation to participate in a specific experiment to determine if the fluctuation in the subject's biomarker level can be altered; and a suggestion or recommendation to perform another measurement at a later time.

Clause 15. The method of clause 1, wherein determining the change in the biomarker during the time interval further comprises determining the relative change in the measurement of the surrogate for the biomarker during the time interval and equating the change in the biomarker to the relative change in the measurement of the surrogate for the biomarker.

Clause 16. The method of clause 8, wherein the source of illumination emits light at a wavelength or wavelengths at which a fat molecule absorbs light.

Clause 17. The method of clause 16, wherein the source of illumination comprises a light emitting diode.

Clause 18. The method of clause 17, wherein the light emitting diode is configured to emit light over a wavelength band.

Clause 19. The method of clause 14, further comprising: based on the determined change in the biomarker during the time interval, generating a suggested change to the subject's diet, exercise regimen or aspect of their lifestyle; and generating the user interface on the device to present the suggested change to a user of the device.

Clause 20. The method of claim 4, further comprising determining a measurement of the fat molecules in the blood twice in a period of a day and based on a difference between the two measurements, determining whether to recommend a change to the subjects' diet, exercise regimen, or aspect of their lifestyle.

Clause 21. The method of clause 1, further comprising receiving information describing the subject's current diet, exercise regimen, or lifestyle, and based on the change in the biomarker during the time interval, recommending a change to a characteristic of the current diet, exercise regimen, or lifestyle.

Clause 22. The method of clause 1, varying a force applied to the subject's tissue to alter a volume of blood within the tissue.

Clause 23. The method of clause 11, further comprising varying a force applied to the subject's finger to alter the volume of blood in a region of the subject's finger during the first or the second non-invasive measurement.

Clause 24. The method of clause 23, wherein the force applied to the subject's finger is varied by one or more of an actuator configured to increase or decrease a force applied to the subject's finger by a finger clip, a cuff arranged on the subject's finger and a pressure regulator configured to vary a pressure applied to the subject's finger by the cuff, and a vacuum chamber placed over a section of the subject's finger and a vacuum pump configured to vary a pressure applied to the section of the subject's finger in contact with the vacuum chamber.

Clause 25. An apparatus comprising: a processor configured with instructions for performing a first non-invasive measurement of a surrogate for a biomarker in a subject's blood at a first time; performing a second non-invasive measurement of the surrogate for the biomarker in the subject's blood at a second time; determining a change in the measurement of the surrogate for the biomarker during a time interval defined by the first time and the second time; and based on the determined change in the measurement of the surrogate for the biomarker, determining a change in the biomarker during the time interval.

Clause 26. The apparatus of clause 25, wherein performing the first non-invasive measurement of the surrogate comprises performing a measurement of a signal that comprises a contribution from the surrogate and the method further comprises determining a component of the measured signal that varies in time in a manner that is correlated with the subject's heartbeat.

Clause 27. The apparatus of clause 25, wherein performing the second non-invasive measurement of the surrogate comprises performing a measurement of a signal that includes a contribution from the surrogate and the method further comprises determining a component of the measured signal that varies in time in a manner that is correlated with the subject's heartbeat.

Clause 28. The apparatus of clause 25, wherein the measurement of the signal is a measurement of fat molecules, and the measurement of the surrogate is a measurement of the fat molecules in the blood.

Clause 29. The apparatus of clause 28, wherein the biomarker is triglyceride.

Clause 30. The apparatus of clause 29, wherein performing the non-invasive measurement comprises collecting spectroscopic data that includes a contribution from the surrogate for the biomarker.

Clause 31. The apparatus of clause 28, wherein collecting spectroscopic data further comprising using a near infrared spectroscopy device to collect the spectroscopic data.

Clause 32. The apparatus of clause 25, wherein the near infrared spectroscopy device comprises a source of illumination, a sensor or detector, and a controller configured to operate the source of illumination and to receive signals from the sensor or detector.

Clause 33. The apparatus of clause 25, wherein the non-invasive measurement comprises a transdermal measurement.

Clause 34. The apparatus of clause 33, wherein performing the non-invasive measurement further comprises: configuring a window that is substantially transparent to near infrared radiation to enable its placement against the skin of the subject; operating a source of illumination to generate near infrared radiation; directing the generated radiation to pass through the window and intersect the skin of the subject; and sensing or detecting a component of the generated radiation after the radiation passes from the subject's skin back through the window.

Clause 35. The apparatus of clause 34, wherein the window is configured to be placed against the subject's finger, arm, or forehead, and further, wherein the source of illumination and the sensor or detector are arranged to be on the same side of the subject's finger, arm, or forehead.

Clause 36. The apparatus of clause 33, wherein performing the non-invasive measurement further comprises: configuring a first window that is substantially transparent to near infrared radiation to enable its placement against the skin of the subject; operating a source of illumination to generate near infrared radiation; directing the generated radiation to pass through the first window and intersect the skin of the subject; and sensing or detecting a component of the generated radiation after the radiation passes from the subject's skin through a second window.

Clause 37. The apparatus of clause 36, wherein the first and second windows are configured to be placed on opposite sides of the subject's finger or earlobe.

Clause 38. The apparatus of clause 25, wherein the processor is further configured with instructions for presenting one or more of the following on a user interface of a device: an alert that the subject's biomarker levels have increased or decreased during the time interval; a suggestion or recommendation to participate in a specific experiment to determine if the fluctuation in the subject's biomarker level can be altered; and a suggestion or recommendation to perform another measurement at a later time.

Clause 39. The apparatus of clause 25, wherein determining the change in the biomarker during the time interval further comprises determining the relative change in the measurement of the surrogate for the biomarker during the time interval and equating the change in the biomarker to the relative change in the measurement of the surrogate for the biomarker.

Clause 40. The apparatus of clause 32, wherein the source of illumination emits light at a wavelength or wavelengths at which a fat molecule absorbs light.

Clause 41. The apparatus of clause 40, wherein the source of illumination comprises a light emitting diode.

Clause 42. The apparatus of clause 41, wherein the light emitting diode is configured to emit light over a wavelength band.

Clause 43. The apparatus of clause 38, wherein the processor is further configured for: based on the determined change in the biomarker during the time interval, generating a suggested change to the subject's diet, exercise regimen or aspect of their lifestyle; and generating the user interface on the device to present the suggested change to a user of the device.

Clause 44. The apparatus of clause 28, wherein the processor is further configured for determining a measurement of the fat molecules in the blood twice in a period of a day and based on a difference between the two measurements, determining whether to recommend a change to the subjects' diet, exercise regimen, or aspect of their lifestyle.

Clause 45. The apparatus of clause 25, wherein the processor is further configured for receiving information describing the subject's current diet, exercise regimen, or lifestyle, and based on the change in the biomarker during the time interval, recommending a change to a characteristic of the current diet, exercise regimen, or lifestyle.

Clause 46. The apparatus of clause 25, further comprising a means for varying a force applied to the subject's tissue to alter a volume of blood within the tissue.

Clause 47. The apparatus of clause 35, further comprising means for varying a force applied to the subject's finger to alter the volume of blood in a region of the subject's finger during the first or the second non-invasive measurement.

Clause 48. The apparatus of clause 47, wherein the force applied to the subject's finger is varied by one or more of an actuator configured to increase or decrease a force applied to the subject's finger by a finger clip, a cuff arranged on the subject's finger and a pressure regulator configured to vary a pressure applied to the subject's finger by the cuff, and a vacuum chamber placed over a section of the subject's finger and a vacuum pump configured to vary a pressure applied to the section of the subject's finger in contact with the vacuum chamber.

Clause 49. An apparatus, comprising: a spectrometer configured to illuminate a region on a subject's skin with near infrared radiation; and a processor operatively coupled to the spectrometer and configured with instructions to cause the apparatus to generate spectroscopic data from signals detected after transmission of the radiation through the subject's skin or from backscatter from a constituent of the subject's blood; and determine a value for a surrogate for a biomarker from the spectroscopic data.

Clause 50. The apparatus of clause 49, wherein determining the value for the surrogate for the biomarker comprises measuring a signal that comprises a contribution from the surrogate and determining a component of the measured signal that varies in time in a manner that is correlated with the subject's heartbeat.

Clause 51. The apparatus of clause 50, wherein the measurement of the signal is a measurement of fat molecules, and the measurement of the surrogate is a measurement of the fat molecules in the blood.

Clause 52. The apparatus of clause 50, wherein the biomarker is triglyceride.

Clause 53. The apparatus of clause 49, wherein the spectrometer further comprises a source of illumination, a sensor or detector, and a controller configured to operate the source of illumination and to receive signals from the sensor or detector.

Clause 54. The apparatus of clause 49, further comprising a first window configured to be placed against the subject's skin and through which the near infrared radiation passes to illuminate the subject's skin, and further wherein the spectroscopic data is generated from signals detected after backscatter from a constituent of the subject's blood.

Clause 55. The apparatus of clause 49, further comprising a first window and a second window with both windows configured to be placed against the subject's skin, wherein the near infrared radiation passes through the first window to illuminate the subject's skin, and further wherein the spectroscopic data is generated from signals detected after transmission of the radiation through the second window.

Clause 56. The apparatus of clause 49, wherein the processor is further configured with instructions to cause the apparatus to: determine a change in the value of the surrogate for the biomarker during a time interval; based on the determined change in the value of the surrogate for the biomarker, determine a change in the biomarker during the time interval; based on the determined change in the biomarker during the time interval, generate a suggested change to the subject's diet, exercise regimen or aspect of their lifestyle; and generate a user interface on a device to present the suggested change to a user of the device.

Clause 57. The apparatus of clause 56, wherein determining the change in the biomarker during the time interval further comprises determining the relative change in the measurement of the surrogate for the biomarker during the time interval and equating the change in the biomarker to the relative change in the measurement of the surrogate for the biomarker.

Clause 58. The apparatus of clause 49, further comprising means for varying a force applied to a subject's tissue to alter a volume of blood in the tissue.

Clause 59. The apparatus of clause 49, further comprising means for varying a force applied to a subject's finger to alter a volume of blood in a region of the subject's finger during prior to or during generation of the spectroscopic data.

Clause 60. The apparatus of clause 58, wherein the force applied to the subject's finger is varied by one or more of an actuator configured to increase or decrease a force applied to the subject's finger by a finger clip, a cuff arranged on the subject's finger and a pressure regulator configured to vary a pressure applied to the subject's finger by the cuff, and a vacuum chamber placed over a section of the subject's finger and a vacuum pump configured to vary a pressure applied to the section of the subject's finger in contact with the vacuum chamber.

Clause 61. A set of one or more non-transitory computer-readable media comprising a set of computer-executable instructions that when executed by one or more programmed electronic processors, cause the processors to perform a first non-invasive measurement of a surrogate for a biomarker in a subject's blood at a first time; perform a second non-invasive measurement of the surrogate for the biomarker in the subject's blood at a second time; determine a change in the measurement of the surrogate for the biomarker during a time interval defined by the first time and the second time; and based on the determined change in the measurement of the surrogate for the biomarker, determine a change in the biomarker during the time interval.

Clause 62. The set of one or more non-transitory computer-readable media of clause 60, wherein performing the first non-invasive measurement of the surrogate comprises performing a measurement of a signal that comprises a contribution from the surrogate and determining a component of the measured signal that varies in time in a manner that is correlated with the subject's heartbeat.

Clause 63. The set of one or more non-transitory computer-readable media of clause 61, wherein performing the second non-invasive measurement of the surrogate comprises performing a measurement of a signal that includes a contribution from the surrogate and determining a component of the measured signal that varies in time in a manner that is correlated with the subject's heartbeat.

Clause 64. The set of one or more non-transitory computer-readable media of clause 61, wherein the measurement of the signal is a measurement of fat molecules, and the measurement of the surrogate is a measurement of the fat molecules in the blood.

Clause 65. The set of one or more non-transitory computer-readable media of clause 63, wherein the biomarker is triglyceride.

Clause 66. The set of one or more non-transitory computer-readable media of clause 60, wherein performing the non-invasive measurement comprises collecting spectroscopic data that includes a contribution from the surrogate for the biomarker.

Clause 67. The set of one or more non-transitory computer-readable media of clause 60, further comprising instructions that cause the processors to: present at least one suggested experiment regarding nutrition, an exercise regimen, or lifestyle to a user via a graphical user interface of a user device; receive a selection by the user device of the suggested experiment by the user; present a prompt on the user device based on the selected experiment to remind the user to perform a change to their nutrition, an exercise regimen, or lifestyle; present a prompt on the user device to the user to perform the first or second non-invasive measurement; process, in the user device or in another computing device, spectroscopic data corresponding to the first or second non-invasive measurement; and present results of the selected experiment, based at least in part on the spectroscopic data, via a graphical user interface of the user device, wherein the results comprise a change in a value of the surrogate for the biomarker over a time interval.

Clause 68. The set of one or more non-transitory computer-readable media of clause 60, further comprising instructions that cause the processors to vary a force applied to a subject's tissue to alter a volume of blood in the tissue.

Clause 69. The set of one or more non-transitory computer-readable media of clause 60, further comprising instructions that cause the processors to vary a force applied to a subject's finger to alter a volume of blood in a region of the subject's finger during the first or the second non-invasive measurement.

Clause 70. The set of one or more non-transitory computer-readable media of clause 65, wherein the force applied to the subject's finger is varied by one or more of an actuator configured to increase or decrease a force applied to the subject's finger by a finger clip, a cuff arranged on the subject's finger and a pressure regulator configured to vary a pressure applied to the subject's finger by the cuff, and a vacuum chamber placed over a section of the subject's finger and a vacuum pump configured to vary a pressure applied to the section of the subject's finger in contact with the vacuum chamber.

Clause 71. An apparatus comprising: a processor configured with instructions for presenting at least one lifestyle change experiment to a user via a graphical user interface of the apparatus; receiving a selection of the lifestyle change experiment by the user; presenting a prompt on the apparatus based on the selected experiment to remind the user to perform a lifestyle change in accordance with the selected experiment; presenting a prompt on the apparatus to the user to perform a measurement of their blood; processing, in the apparatus or in another computing device, spectroscopic data corresponding to the blood measurement; and presenting results of the selected experiment, based at least in part on the spectroscopic data, via a graphical user interface of the apparatus, wherein the results comprise a change in a value of a surrogate for a biomarker over a time interval.

Clause 72. The apparatus of clause 69, wherein the surrogate is a fat level in the blood and the biomarker is a triglyceride level.

Clause 73. The apparatus of clause 69, wherein the prompt instructs the user to perform the blood measurement using a device for performing a non-invasive blood measurement.

Clause 74. The apparatus of clause 71, wherein the device includes a spectrometer for generating the spectroscopic data.

Clause 75. The apparatus of clause 69, wherein the presented results further comprise a change in the biomarker over the time interval and the change in the biomarker is determined from the relative change in the measurement of the surrogate for the biomarker during the time interval and equating the change in the biomarker to the relative change in the measurement of the surrogate for the biomarker.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof

What is claimed is:

1. A method, comprising:
performing a first non-invasive measurement of a surrogate for a biomarker in a subject's blood at a first time;
performing a second non-invasive measurement of the surrogate for the biomarker in the subject's blood at a second time;
determining a change in the measurement of the surrogate for the biomarker during a time interval defined by the first time and the second time; and
based on the determined change in the measurement of the surrogate for the biomarker, determining a change in the biomarker during the time interval.

2. The method of claim 1, wherein performing the first non-invasive measurement of the surrogate comprises performing a measurement of a signal that comprises a contribution from the surrogate and the method further comprises determining a component of the measured signal that varies in time in a manner that is correlated with the subject's heartbeat.

3. The method of claim 2, wherein performing the second non-invasive measurement of the surrogate comprises performing a measurement of a signal that includes a contribution from the surrogate and the method further comprises determining a component of the measured signal that varies in time in a manner that is correlated with the subject's heartbeat.

4. The method of claim 2, wherein the measurement of the signal comprises a measurement of fat molecules, and the measurement of the surrogate comprises a measurement of the fat molecules in the blood.

5. The method of claim 4, wherein the biomarker comprises one or more triglycerides.

6. The method of claim 1, wherein performing the non-invasive measurement comprises collecting spectroscopic data that includes a contribution from the surrogate for the biomarker.

7. The method of claim 6, further comprising using a near infrared spectroscopy device to collect the spectroscopic data.

8. The method of claim 7, wherein the near infrared spectroscopy device comprises a source of illumination, a sensor or detector, and a controller configured to operate the source of illumination and to receive signals from the sensor or detector.

9. The method of claim 1, wherein the non-invasive measurement comprises a transdermal measurement.

10. The method of claim 9, wherein performing the non-invasive measurement further comprises:
configuring a window that is substantially transparent to near infrared radiation to enable its placement against the skin of the subject;
operating a source of illumination to generate near infrared radiation;
directing the generated radiation to pass through the window and intersect the skin of the subject; and
measuring a component of the generated radiation after the radiation passes from the subject's skin back through the window.

11. The method of claim 10, wherein the window is configured to be placed against the subject's finger, arm, or forehead, and further, wherein the source of illumination and the sensor or detector are arranged to be on the same side of the subject's finger, arm, or forehead.

12. The method of claim 9, wherein performing the non-invasive measurement further comprises:
configuring a first window that is substantially transparent to near infrared radiation to enable its placement against the skin of the subject;
operating a source of illumination to generate near infrared radiation;
directing the generated radiation to pass through the first window and intersect the skin of the subject; and
measuring a component of the generated radiation after the radiation passes from the subject's skin through a second window.

13. The method of claim 12, wherein the first and second windows are configured to be placed on opposite sides of the subject's finger or earlobe.

14. The method of claim 1, further comprising presenting one or more of the following on a user interface of a device:
an alert that the subject's biomarker levels have increased or decreased during the time interval;
a suggestion or recommendation to participate in a specific experiment to determine if the fluctuation in the subject's biomarker level can be altered; and
a suggestion or recommendation to perform another measurement at a later time.

15. The method of claim 1, wherein determining the change in the biomarker during the time interval further comprises determining the relative change in the measurement of the surrogate for the biomarker during the time interval and equating the change in the biomarker to the relative change in the measurement of the surrogate for the biomarker.

16. The method of claim 8, wherein the source of illumination emits light at a wavelength or wavelengths at which a fat molecule absorbs light.

17. The method of claim 16, wherein the source of illumination comprises a light emitting diode.

18. The method of claim 17, wherein the light emitting diode is configured to emit light over a wavelength band.

19. The method of claim 14, further comprising:
based on the determined change in the biomarker during the time interval, generating a suggested change to the subject's diet, exercise regimen or aspect of their lifestyle; and
generating the user interface on the device to present the suggested change to a user of the device.

20. The method of claim 4, further comprising determining a measurement of the fat molecules in the blood twice in a period of a day and based on a difference between the two measurements, determining whether to recommend a change to the subjects' diet, exercise regimen, or aspect of their lifestyle.
